(19) United States
(12) Patent Application Publication
(10) Pub. No.: US 2008/0231343 A1
(43) Pub. Date: Sep. 25, 2008

(54) Method and System for Vibration Sensing Power Management

(75) Inventors: Nigel Leigh, Christchurch (NZ); Carl Omundsen, Christchurch (NZ)

Correspondence Address: PITTS AND BRITTIAN P C, P O BOX 51295, KNOXVILLE, TN, 37950-1295, US

(73) Assignee: Commtest Instruments, Ltd., Christchurch (NZ)

(21) Appl. No.: 12/050,600

(22) Filed: Mar. 18, 2008

(30) Foreign Application Priority Data
Mar. 19, 2007 (NZ) 553955

(51) Int. Cl. G01N 33/00

(52) U.S. Cl. 327/516; 702/56

(57) ABSTRACT

The invention provides in one aspect a method for vibration sensing. The method comprises powering down at least one electronic component required for recording vibration data; powering down a processor used to carry out one or more steps of the method; measuring the vibration level of a machine in a frequency band of interest; comparing the measured vibration level with a user-selected reference vibration level; and powering up the processor if the measured vibration level is greater than or equal to the reference vibration level. In another aspect the invention provides a system for vibration sensing.

### CROSS-REFERENCE TO RELATED APPLICATIONS

This application claims the benefit of the Provisional Specification filed on Mar. 19, 2007 with the Intellectual Property Office of New Zealand having Letters Patent number 553955.

### STATEMENT REGARDING FEDERALLY SPONSORED RESEARCH OR DEVELOPMENT

Not Applicable

### FIELD OF THE INVENTION

The present invention relates to a method and system for vibration sensing power management. In particular, but not exclusively, the present invention relates to a method and system for vibration sensing where components used for vibration data recording are powered up when the vibration sensed is equal to or above a user selectable reference level.

### BACKGROUND TO THE INVENTION

Condition monitoring by measuring and trending vibration levels and their related frequencies has become the accepted method for preventative maintenance of machinery. The Integrated Circuit Piezoelectric (ICP) accelerometer is the industry sensor of choice, requiring cabling supplying power and signal lines between itself and the monitoring system. The cost of the cabling and its installation normally outweighs the cost of any other part of the monitoring system. A self-powered vibration sensor that can communicate with a monitoring system wirelessly is potentially a more cost-effective solution.

The candidate power sources for a self-powered vibration sensor have a restricted capacity e.g. battery power, power scavenged from light or vibration. Consequently, the circuitry required to capture the vibration data cannot be powered on at all times and the sensor must be powered on periodically. However, when in a powered down state, the sensor may either miss a high vibration event altogether or capture it too late to prevent serious damage to the machine being monitored.

An example prior art vibration monitoring system is described in U.S. Pat. No. 5,854,994 to Canada et al. In that system, a plurality of machine monitors are employed to sense the vibration levels of machinery. As shown in FIG. 3 of the patent, each machine monitor includes at least one sensor and a wireless transmitter to transmit sensor data to a central station. The machine monitors are also self-powered, using, for example, a replaceable D-cell battery. To conserve power, the system of Canada et al. includes a fixed schedule for when the machine monitors are turned on.

In this specification, where reference has been made to patent specifications, other external documents, or other sources of information, this is generally for the purpose of providing a context for discussing the features of the invention. Unless specifically stated otherwise, reference to such external documents or sources of information is not to be construed as an admission that such documents or sources of information in any jurisdiction are prior art, or form part of the common general knowledge in the art.

Given the above background, it is an object of the present invention to either provide a method and system that improves the power consumption and the response to high vibration events of a self-powered vibration sensor or at least provide the public with a useful choice.

### SUMMARY OF THE INVENTION

In one aspect, the present invention broadly relates to a method for vibration sensing comprising: powering down at least one electronic component required for recording vibration data; powering down a processor used to carry out one or more steps of the method; measuring the vibration level of a machine in a frequency band of interest; comparing the measured vibration level with a user-selected reference vibration level; and powering up the processor if the measured vibration level is greater than or equal to the reference vibration level.

Preferably, the method further comprises the steps of once the processor is powered up: powering down at least one component required for measuring the vibration level; and powering down at least one component required for comparing the measured vibration level to a reference vibration level.

Preferably, the method further comprises the step of powering down at least one component required for measuring and powering down the at least one component required for comparing for a certain period of time or until instructed otherwise by a monitoring system.

Preferably, the method further comprises the step of once the processor is powered up: ignoring the result of the comparison of the measured vibration level with the user-selected reference vibration level.

Preferably, the method further comprises the step of ignoring the result of the comparison of the measured vibration level with the user-selected reference vibration level for a certain period of time or until instructed otherwise by a monitoring system.

Preferably, the method further comprises the step of powering up at least one electronic component required for recording vibration data.

Preferably, the method further comprises the step of notifying a monitoring system that the reference vibration level has been matched or exceeded.

Preferably, the method further comprises the step of using the processor to power down the electronic components required for recording vibration data once the data has been captured.

In another aspect, the present invention broadly relates to a method of vibration sensing comprising measuring the vibration level of a machine in a frequency band of interest; comparing the measured vibration level with a user-selected reference vibration level; and powering up a processor or at least one other electronic component required for recording vibration data if the measured vibration level is greater than or equal to the reference vibration level.

Preferably, the method further comprises the step of receiving a user selection of a reference vibration level.

Preferably, the vibration level of the machine is measured using one or more accelerometers to produce acceleration signals.

Preferably, the measured vibration level is an overall vibration velocity value, and the reference vibration level is an overall reference vibration velocity value.

Preferably, the processor, once powered up, powers up further electronic components required for recording vibration data.

Preferably, the electronic components include: a buffer, an amplifier, a low-pass filter and/or an analogue-to-digital converter.

Preferably, the processor, once powered up, notifies a monitoring system of the reference vibration level being matched or exceeded and optionally powers up the other electronic components required for recording vibration data.

Preferably, the at least one electronic component required for measuring vibration data is powered down once the data has been captured.

In a further aspect, the present invention broadly relates to a system for vibration sensing comprising one or more vibration sensing elements to generate electrical vibration signals representing sensed vibrations; a first circuit to generate an overall vibration level based on the electrical vibration signals, the first circuit including a comparator to compare the overall vibration level with the reference vibration level; a reference setting device for a user to set a reference vibration level; a second circuit to record the electrical vibration signals; and a processor configured to: power down at least one component of the second circuit; power down itself; and power up itself if the overall vibration level is greater than or equal to the reference vibration level as determined by the comparator from the first circuit.

Preferably, the processor, once powered up, is configured to disable the first circuit, the reference setting device and the comparator.

Preferably, the disabling is achieved by powering down at least one component in the first circuit, the reference setting device, and the comparator.

Preferably, the first circuit, the reference setting device and comparator are disabled for a certain period of time or until instructed otherwise by the monitoring system.

Preferably, the disabling is achieved by ignoring the result of the comparison of the overall vibration level with the reference vibration level.

Preferably, the comparison of the overall vibration level with the reference vibration level is ignored for a certain period of time or until instructed otherwise by a monitoring system.

Preferably, the processor, once powered up, is configured to power up all of the components in the second circuit to record vibration data.

Preferably, the processor, once powered up, is configured to send a signal to a monitoring system to notify the monitoring system that the reference level was matched or exceeded.

Preferably, the at least one electronic component in the second circuit is powered down once the data has been captured.

Preferably, the first circuit and the second circuit share one of the vibration sensing elements.

In a further aspect, the present invention broadly relates to a system for vibration sensing comprising one or more vibration sensing elements that generate electrical vibration signals representing sensed vibrations; a first circuit that generates an overall vibration level based on the electrical vibration signals; a reference setting device that is used to variably set or select a reference vibration level; a comparator that compares the overall vibration level with the reference vibration level; and a second circuit that is powered up to record the electrical vibration signals if the overall vibration level is greater than or equal to the reference vibration level.

Preferably, the one or more vibration sensing elements are one or more accelerometers.

Preferably, the first circuit generates an overall velocity vibration level.

Preferably, the comparator is connected to: (i) the first circuit to receive the overall velocity vibration level and (ii) a digital potentiometer as the reference setting device for setting the reference vibration level.

Preferably, the second circuit comprises: a buffer, an amplifier, a low-pass filter and an analogue-to-digital converter.

Preferably, the system is formed as part of a self-powered vibration sensor.

Preferably, the system includes a wireless transmitting device for Tirelessly transmitting the recorded vibration signals to a monitoring system.

Preferably, the at least one component in the second circuit is switched off to power down the second circuit.

Preferably, the first circuit and the second circuit share one of the vibration sensing elements.

The term ‘comprising’ as used in this specification means ‘consisting at least in part of’, that is to say when interpreting statements in this specification which include that term, the features, prefaced by that term in each statement, all need to be present but other features can also be present. Related terms such as ‘comprise’ and ‘comprised’ are to be interpreted in similar manner.

As used herein the term “and/or” means “and” or “or”, or both.

As used herein “(s)” following a noun means the plural and/or singular forms of the noun.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

The invention consists in the foregoing and also envisages constructions of which the following gives examples only.

### BRIEF DESCRIPTION OF THE FIGURES

Preferred forms of the method and system of the present invention will now be described with reference to the accompanying figures in which:

FIG. 1 shows a simplified schematic of the system of the present invention, and

FIG. 2 shows a detailed schematic of the system of the present invention.

### DETAILED DESCRIPTION OF THE INVENTION

### The System of the Present Invention

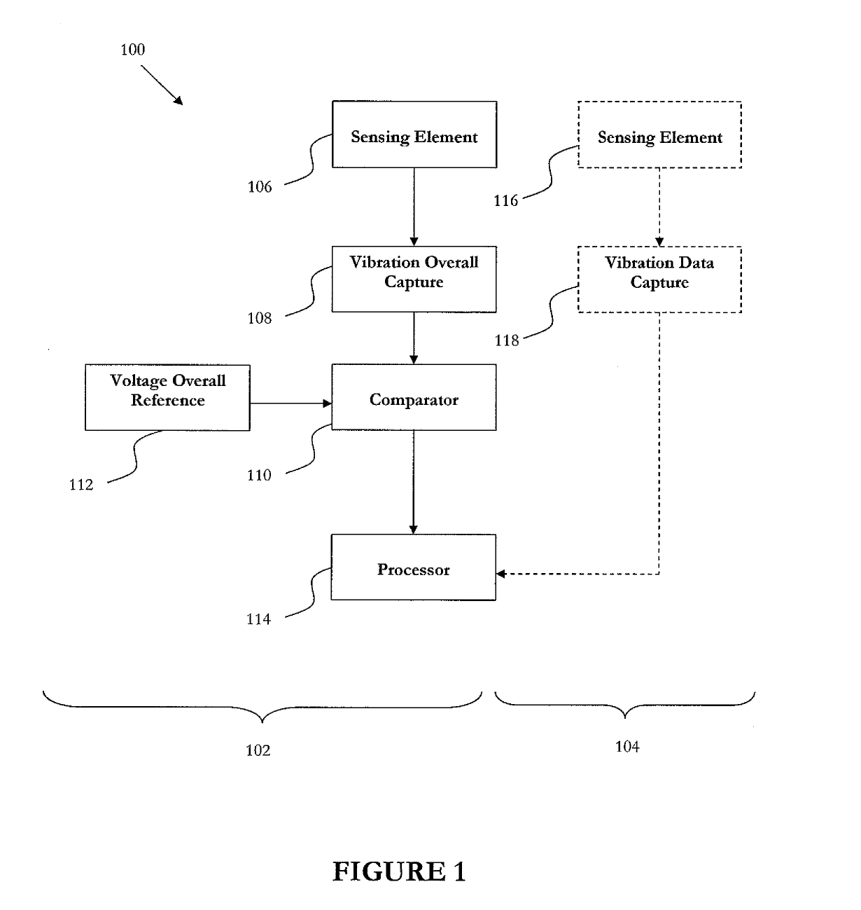

Referring to FIG. 1, a simplified schematic of the preferred form system is shown generally as 100. The system 100 is preferably constructed to form part of a self-powered vibration sensor, which itself forms part of a machinery condition monitoring system.

As illustrated, the system 100 comprises two main circuits: an overall vibration monitoring circuit 102 (shown in solid lines) and a vibration data recording or data capture circuit 104 (shown in dashed lines). In essence, the monitoring circuit 102 monitors the overall vibration level of a machine, while the data capture circuit 104 records the vibrations of the machine when the overall vibration level is greater than or equal to a reference vibration level. The data capture circuit 104 can also be used independently of the monitoring circuit 102 for regular scheduled recordings.

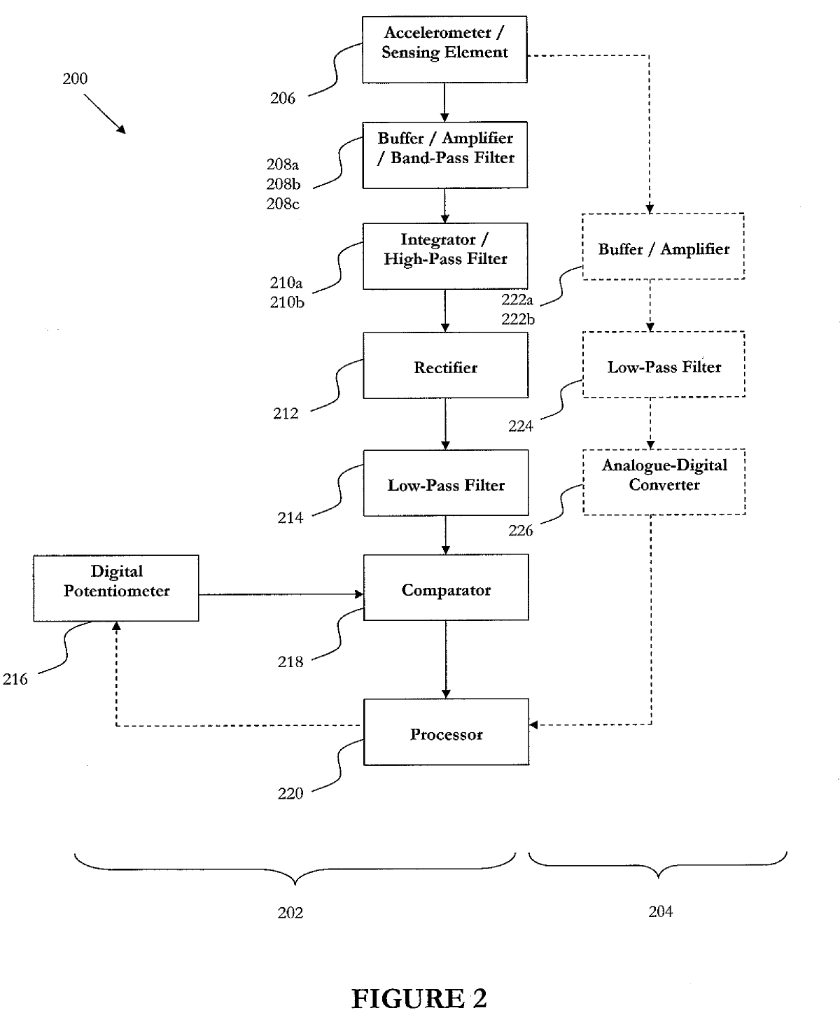

The monitoring circuit 102 includes a vibration sensing element 106. In the preferred form, the sensing element 106 is an accelerometer. Where necessary or desired, velocity or displacement sensing elements may be used instead. All that is required of the sensing element is the ability to sense and represent mechanical vibrations of a machine as electrical vibration signals. Those electrical vibration signals would then be processed by an overall capture component 108 to produce an overall vibration level. Skilled persons will appreciate that determining an overall vibration level can be done using many techniques. One non-limiting example technique is determining the average of the vibration signals. This will be described in more detail with reference to FIG. 2.

Once the overall vibration level is determined, it is compared with a reference vibration level in a comparator 1 10. The reference vibration level is a value representing the threshold at which vibration data recording should be made. It is selected or specified by a user and is implemented using a reference setting device 112. If the overall vibration level is less than the reference vibration level, the system 100 is maintained at a low-power or standby state. This is done by a processor 114, which is configured to power down at least one component of the data capture circuit 104. To further conserve power, the processor 114 is also configured to power itself down. If, on the other hand, the overall vibration level is greater than or equal to the reference vibration level, the comparator 110 sends a signal to the processor 114 to power on the processor. The processes of sensing vibrations, determining an overall vibration level and comparing the overall vibration level with a user-selected reference vibration level is ultimately controlled by the processor. Once powered on by the comparator's signal, the processor 114 preferably performs one or more pre-programmed actions. This may include sending a warning message to a monitoring system and fully powering on the data capture circuit 104 to capture vibration data. Once data has been captured, the processor 114 preferably powers down at least one component of the data capture circuit 104.

To ensure the process above is not repeated continuously, the processor 114 may preferably disable the monitoring circuit, the reference setting device and/or the comparator. This could be done, for instance, by powering off one or more components of the monitoring circuit 102, the reference setting device and the comparator, or just ignoring the comparator's signal until such a time that the monitoring circuit 102 is requested to be armed again by the monitoring system, or until a time period elapses.

As with the monitoring circuit 102, the data capture circuit 104 also includes a sensing element 116. Although separate sensing elements are shown for the monitoring circuit 102 and the data capture circuit 104, it is possible to instead use a single sensing element for both circuits 102 and 104.

The data capture circuit 104 also includes a data capture component 118 to capture vibration data, typically as a digitally-sampled vibration signal or a single overall value.

With the above arrangement, the preferred form system of the present invention allows more power to be conserved in a self-powered vibration sensor. In particular, the present invention divides the components of the sensor into components that are required to be powered at all times and components that are only required to be powered intermittently. Those components that are required to be powered at all times are the components in the monitoring circuit 102, which are separated from the components that only need to powered intermittently, which are the components of the data capture circuit 104. Where data capture is not required, for instance where the machine is running normally with vibrations below the reference level, the data capture circuit 104 is powered down or put into a standby state by the monitoring circuit 102. Once the vibrations meet or exceed the reference level, the data capture circuit 104 is powered on and the system 100 is woken up from its low-power or standby state by the monitoring circuit 102. Immediate capture of vibration data then follows. It can therefore be said that the monitoring circuit functions as a power management system for the data capture circuit 104 and the system 100 as a whole.

A more detailed explanation of the preferred form system will now be provided with reference to FIG. 2. The system, which is again preferably constructed as part of a self-powered vibration sensor, is shown generally as 200 in the figure. As before, the system 200 includes two main circuits: a monitoring circuit 202 (shown in solid lines) and a data capture circuit 204 (shown in dashed lines). When the vibrations sensed by the monitoring circuit 202 are below a selected reference, the system 200 enters into a low-power or standby mode by powering down the data capture circuit 204. Preferred components to do this will be described below.

The monitoring circuit 202, as described earlier, includes an accelerometer 206 as a sensing element. The accelerometer converts mechanical vibrations into electrical signals representing the acceleration of the mechanical vibrations. The monitoring circuit 202 also includes a buffer 208a, an amplifier 208b and a bass-pass filter 208c. The band-pass filter 208c is preferably tuned to the 10-1000 Hz band. There is also an integrator 210a, which converts the acceleration signals into velocity signals, and a high-pass filter 210b to remove any signal DC offset. Since the integrator 210a is inherently a low-pass filter, the integrator and high-pass filter combination effectively provides a second stage of band-pass filtering.

A rectifier 212 is used in the monitoring circuit 202 to average the velocity signals from the integrator and high-pass filter combination. The averaged velocity signal is then filtered using a low-pass filter 214. This then produces an overall velocity level representing the vibrations sensed. As noted earlier, this technique is one of many ways in which to obtain an overall vibration signal; skilled persons will appreciate that the present invention need not be limited to the comparison of velocity signals or an average level of the measured vibration signals.

The monitoring circuit 202 also includes a digital potentiometer 216 to set a reference velocity level. The reference velocity level will be selected or specified by a user, typically via the monitoring system. Based on the level selected or specified by the user, the digital potentiometer 216 provides a changeable reference level for use by a comparator 218. The potentiometer's ability to provide a changeable reference level also allows the calibration of the velocity overall amplitudes.

The comparator 218 is used in the monitoring circuit 202 to compare the overall velocity level and the reference velocity level. If the overall velocity level is greater than or equal to the reference velocity level, the comparator 218 generates a signal that is sent to an interrupt pin of the system's processor 220. The processor 220 then powers itself up and the data capture circuit 204 from its low-power mode and begins the process of capturing vibration data. As an alternative or additional step, the processor 220 may send a warning message (which may be a simple signal or a complex message) to a monitoring system.

The data capture circuit 204 includes a buffer 222a and an amplifier 222b that, once powered, respectively buffer and amplify the electrical vibration signals from the accelerometer 206. The data capture circuit 204 also includes a low-pass filter 224 to eliminate aliasing and an analogue-to-digital converter 226 to convert the electrical vibration signals into digital signals. Once converted, the digital signals are captured by the processor 220.

Given the above, it will be clear to skilled persons that it is desirable for the system of the present invention to be used as part of a self-powered vibration sensor that requires low power consumption due to a restricted power supply and that has an on-state that consumes too much power for continuous operation. Instead of the sensor having to supply full power to all of its circuitry continuously, it predominately conserves power in a low-power state and only fully powers itself on when it needs to. Having the components described above, the present invention allows the construction of a low-power monitoring circuit (which draws only 20 μA, for instance) that may remain powered on to manage the power consumption of a data recording circuit, and, in doing so, manage the power consumption of the self-powered sensor as a whole.

### The Method of the Present Invention

As described in the Summary of the Invention, an aspect of the present invention is a method for vibration sensing. In one preferred form, the method begins with the step of a user selecting a reference vibration level. It is, however, not necessary for the reference vibration level to be selected for every iteration of the method of the present invention. Where a reference level has been previously set by the user, and where the user desires to make no change to that setting, it is possible for the method to be carried out based on the previous reference level setting.

The method also includes the steps of measuring the vibration level in a frequency band of interest using a first circuit, and comparing the measured vibration level with the reference vibration level. In the preferred form, the measured vibration level is an overall velocity value, and the reference vibration level is a reference velocity value. To generate the overall velocity value, the first circuit, which is similar to the monitoring circuit described earlier, preferably comprises: a buffer, an amplifier, a band-pass filter, a high-pass filter, an integrator, a rectifier and a low-pass filter.

If the measured vibration level is equal to or greater than the reference vibration level, the preferred form method proceeds to power up a processor or at least one component of the second circuit for recording vibration data. The second circuit is synonymous to the data capture circuit described earlier. Once the processor is powered up, the method preferably includes the step of using the processor to power up other components in the second circuit. Alternatively or additionally, the method may include the step of notifying a monitoring system, using the processor, of the reference vibration level being matched or exceeded.

The method may also begin with the steps of powering down at least one electronic component required for recording vibration data, and powering down a processor used to carry out one or more steps of the method. This allows power conservation from the start of the method. The method then measures the vibration level of a machine in a frequency band of interest and compares the measured vibration level with a user-selected reference vibration level. If the measured vibration level is greater than or equal to the reference vibration level, the method proceeds to power up the processor.

Once the processor is powered up, the steps of measuring the vibration level and comparing it to a reference vibration level are preferably disabled. Disabling can be achieved by powering down at least one component required for measuring the vibration level and/or for performing the comparison. Alternatively or additionally, disabling can be achieved by ignoring the result of the comparison. The disablement of the steps above may remain for a certain period of time or until instructed otherwise by a monitoring system.

Where the components of the second circuit are powered up following the comparison step, or where the processor powers up the components of the second circuit, the method preferably includes the step of using the processor to power down the components of the second circuit once vibration data has been captured.

The foregoing describes the invention including preferred forms thereof. Alterations and modifications as will be obvious to those skilled in the art are intended to be incorporated within the scope hereof. For instance, although the data capture circuit has been described to be powered up when the overall vibration level is equal to or greater than the reference vibration level, it is possible to instead design the system such that the data capture circuit is powered up when the overall vibration level comes within a certain tolerance of the reference vibration level. Further, it will be noted that the components and circuits described above can be powered down without being completely switched off. All that is required is for the component or circuit to consume less power than would otherwise be the case. Also, where a circuit is powered down, it is not essential for every component to be powered down. Rather, the circuit as a whole can be powered down simply by powering down or switching off one or more components in the circuit.

Having thus described the aforementioned invention, what is claimed is:

1. A method for vibration sensing comprising:
powering down at least one electronic component required for recording vibration data;
powering down a processor used to carry out one or more steps of the method;
measuring the vibration level of a machine in a frequency band of interest;
comparing the measured vibration level with a user-selected reference vibration level; and
powering up the processor if the measured vibration level is greater than or equal to the reference vibration level.

2. The method of claim 1 further comprising, once the processor is powered up:
powering down at least one component required for measuring the vibration level; and
powering down at least one component required for comparing the measured vibration level to a reference vibration level.

3. The method of claim 2 comprising powering down the at least one component required for measuring and powering down the at least one component required for comparing for a certain period of time or until instructed otherwise by a monitoring system.

4. The method of claim 1 further comprising, once the processor is powered up: ignoring the result of the comparison of the measured vibration level with the user-selected reference vibration level.

5. The method of claim 4 comprising ignoring the result of the comparison of the measured vibration level with the user-selected reference vibration level for a certain period of time or until instructed otherwise by a monitoring system.

6. The method of claim 1 further comprising powering up at least one electronic component required for recording vibration data.

7. The method of claim 1 further comprising notifying a monitoring system that the reference vibration level has been matched or exceeded.

8. The method of claim 1 further comprising using the processor to power down the electronic components required for recording vibration data once the data has been captured.

9. A method for vibration sensing comprising:
measuring the vibration level of a machine in a frequency band of interest;
comparing the measured vibration level with a user-selected reference vibration level; and
powering up a processor or at least one other electronic component required for recording vibration data if the measured vibration level is greater than or equal to the reference vibration level.

10. The method of claim 9 further comprising receiving a user selection of a reference vibration level.

11. The method of claim 9 wherein the vibration level of the machine is measured using one or more accelerometers to produce acceleration signals.

12. The method of claim 9 wherein the measured vibration level is an overall vibration velocity value, and the reference vibration level is an overall reference vibration velocity value.

13. The method of claim 9 wherein the processor, once powered up, powers up further electronic components required for recording vibration data.

14. The method of claim 13 wherein the further electronic components include: a buffer, an amplifier, a low-pass filter and/or an analogue-to-digital converter.

15. The method of claim 9 wherein the processor, once powered up, notifies a monitoring system of the reference vibration level being matched or exceeded and optionally powers up the other electronic components required for recording vibration data.

16. The method of claim 9 wherein at least one electronic component required for measuring vibration data is powered down once the data has been captured.

17. A system for vibration sensing comprising:
one or more vibration sensing elements to generate electrical vibration signals representing sensed vibrations;
a first circuit to generate an overall vibration level based on the electrical vibration signals, the first circuit including a comparator to compare the overall vibration level with the reference vibration level;
a reference setting device for a user to set a reference vibration level;
a second circuit to record the electrical vibration signals; and
a processor configured to:
power down at least one component of the second circuit;
power down itself; and
power up itself if the overall vibration level is greater than or equal to the reference vibration level as determined by the comparator from the first circuit.

18. The system of claim 17 wherein the processor, once powered up, is configured to disable the first circuit, the reference setting device and the comparator.

19. The system of claim 18 wherein the disabling is achieved by powering down at least one component in the first circuit, the reference setting device, and the comparator.

20. The system of claim 18 wherein the first circuit, the reference setting device and comparator are disabled for a certain period of time or until instructed otherwise by the monitoring system.

21. The system of claim 17 wherein the processor, once powered up, is configured to power up all of the components in the second circuit to record vibration data.

22. The system of claim 17 wherein the processor, once powered up, is configured to send a signal to a monitoring system to notify the monitoring system that the reference level was matched or exceeded.

23. The system of claim 17 wherein at least one electronic component in the second circuit is powered down once the data has been captured.

24. The system of claim 17 wherein the first circuit and the second circuit share one of the vibration sensing elements.

25. A system for vibration sensing comprising:
one or more vibration sensing elements that generate electrical vibration signals representing sensed vibrations;
a first circuit that generates an overall vibration level based on the electrical vibration signals;
a reference setting device that is used to variably set or select a reference vibration level;
a comparator that compares the overall vibration level with the reference vibration level; and
a second circuit that is powered up to record the electrical vibration signals if the overall vibration level is greater than or equal to the reference vibration level.

26. The system of claim 25 wherein the one or more vibration sensing elements are one or more accelerometers.

27. The system of claim 25 wherein the first circuit generates an overall velocity vibration level.

28. The system of claim 25 wherein the comparator is connected to: (i) the first circuit to receive the overall velocity vibration level and (ii) a digital potentiometer as the reference setting device for setting the reference vibration level.

29. The system of claim 25 wherein the second circuit comprises: a buffer, an amplifier, a low-pass filter and an analogue-to-digital converter.

30. The system of claim 25 wherein the system is formed as part of a self-powered vibration sensor.

31. The system of claim 25 wherein the system includes a wireless transmitting device for wirelessly transmitting the recorded vibration signals to a monitoring system.

32. The system of claim 25 wherein at least one component in the second circuit is switched off to power down the second circuit.

33. The system of claim 25 wherein the first circuit and the second circuit share one of the vibration sensing elements.
